(19) United States
(12) Patent Application Publication
(10) Pub. No.: US 2008/0208237 A1
(43) Pub. Date: Aug. 28, 2008

(54) Ear piercing device with disposable components

(76) Inventor: Long Sing Lei, Taipei City (TW)

Correspondence Address: PRO-TECHTOR INTERNATIONAL SERVICES, 20775 NORADA CT., SARATOGA, CA, 95070, US

(21) Appl. No.: 11/709,607

(22) Filed: Feb. 22, 2007

(51) Int. Cl. A61B 17/34

(52) U.S. Cl. 606/188

(57) ABSTRACT

An ear piercing device in one embodiment includes a butt shaped housing, a movable jaw carrier including a spring biased grip member projecting from the housing, and a leg projecting upward from a front end, a spring biased trigger, a spring biased slide bar lockingly engaged with the trigger, a first jaw releasably secured to a front end of the housing, a second jaw releasably secured to the leg, and a sterile needle mounted in a front opening of the housing. Positioning the jaws at both sides of the ear, pressing the grip member to cause the jaws to clamp the ear, and pressing down the trigger will unlock the slide bar to strike the needle so that the needle forcefully moves forward along the opening and the first jaw to insert through the ear into the second jaw. Thereafter, the jaws are adapted to dispose.

### BACKGROUND OF THE INVENTION

1. Field of Invention

The invention relates to ear piercing devices and more particularly to such an ear piercing device having disposable components (e.g., jaws contacting ear during piercing operation) so as to reduce the risk of spreading diseases.

2. Description of Related Art

There have been numerous suggestions in prior patents for ear piercing device. For example, U.S. Pat. No. 4,286,600 describes an ear piercing device having a needle for inserting through the tissue of an ear a sterilized thermoplastic sleeve having a first boss on one end and a second boss on the other end formed by heating by means of a heating element (e.g., copper).

However, components such as needle and bolt of the patent may be contaminated during the piercing operation. Also, it is possible that the subsequent process of sterilizing the contaminated needle and bolt is not done completely. Thus, it is highly possible of spreading diseases to a next user if the same device is reused. Hence, it is desirable to provide a novel ear piercing device having disposable components in order to overcome the inadequacies of the prior art.

### SUMMARY OF THE INVENTION

It is therefore one object of the invention to provide an ear piercing device having components contacting ear during piercing operation adapted to dispose so as to reduce the risk of spreading diseases.

It is another object of the invention to provide an ear piercing device capable of precisely inserting through the tissue of an ear a sterile needle.

The above and other objects, features and advantages of the invention will become apparent from the following detailed description taken with the accompanying drawings.

### BRIEF DESCRIPTION OF THE DRAWINGS

FIG. 1 is an exploded view of a preferred embodiment of ear piercing device according to the invention excluding its disposable components;

FIG. 2 is a perspective view of the assembled ear piercing device shown in FIG. 1 with the disposable components to be mounted thereon;

FIG. 3 is a sectional view of the assembled ear piercing device shown in FIG. 1 in a ready to use state;

FIG. 4 is a view similar to FIG. 3 where first and second jaws are mounted;

FIG. 5 is a view similar to FIG. 4 where an ear is clamped between and by the first and second jaws;

FIG. 6 is a view similar to FIG. 5 where the trigger has been pressed down to unlock the slide bar; and

FIG. 7 is a view similar to FIG. 6 where the unlocked slide bar has striken the needle to insert the needle into the tissue of the ear.

### DETAILED DESCRIPTION OF THE INVENTION

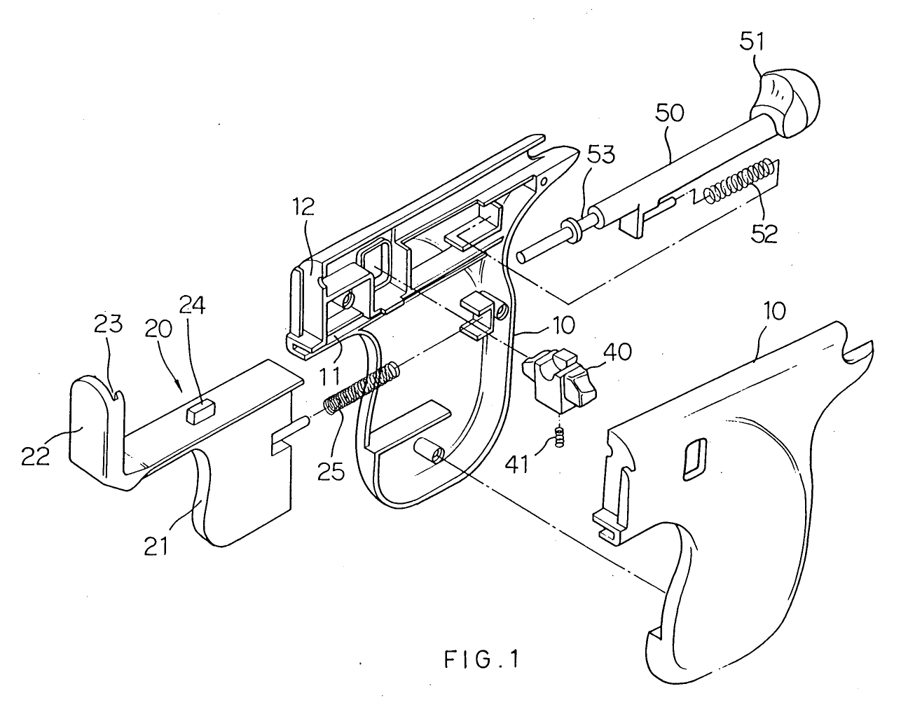

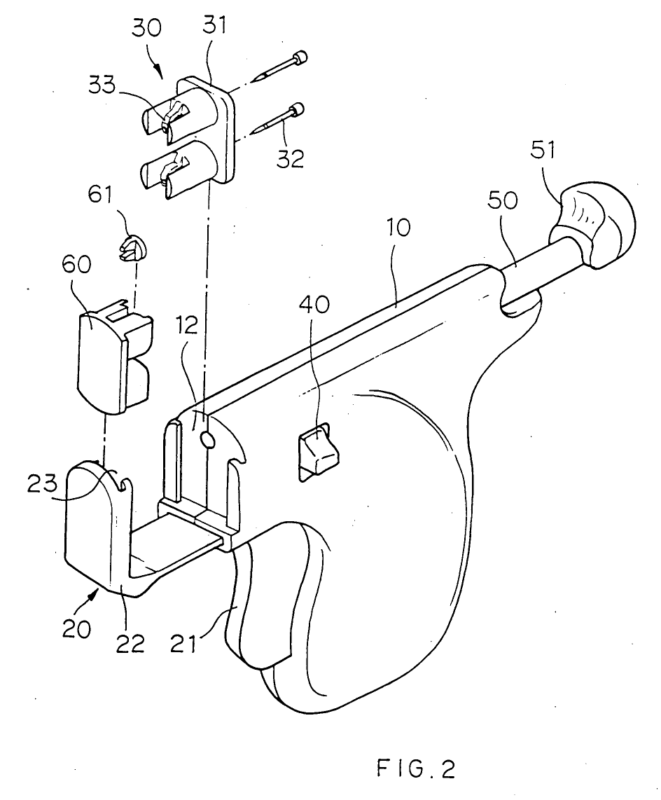

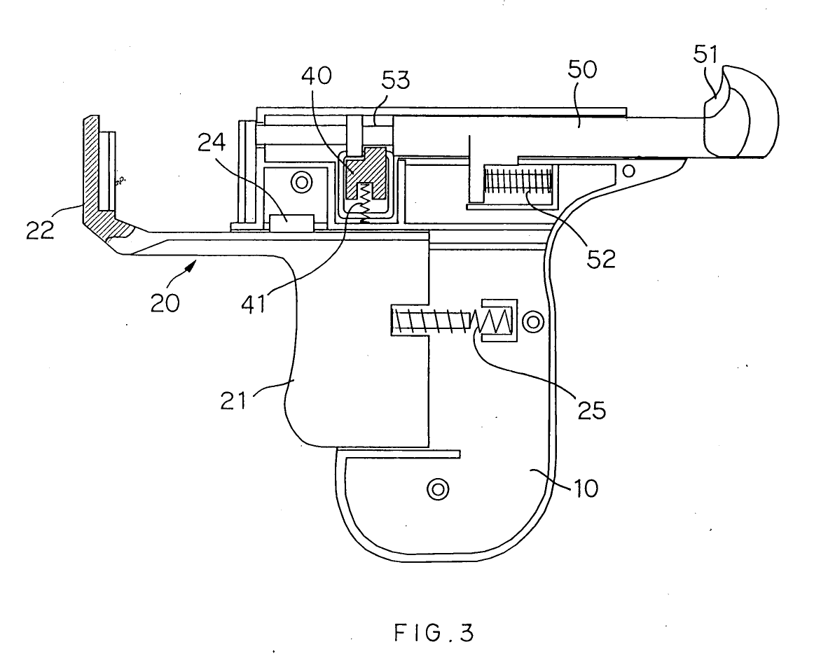

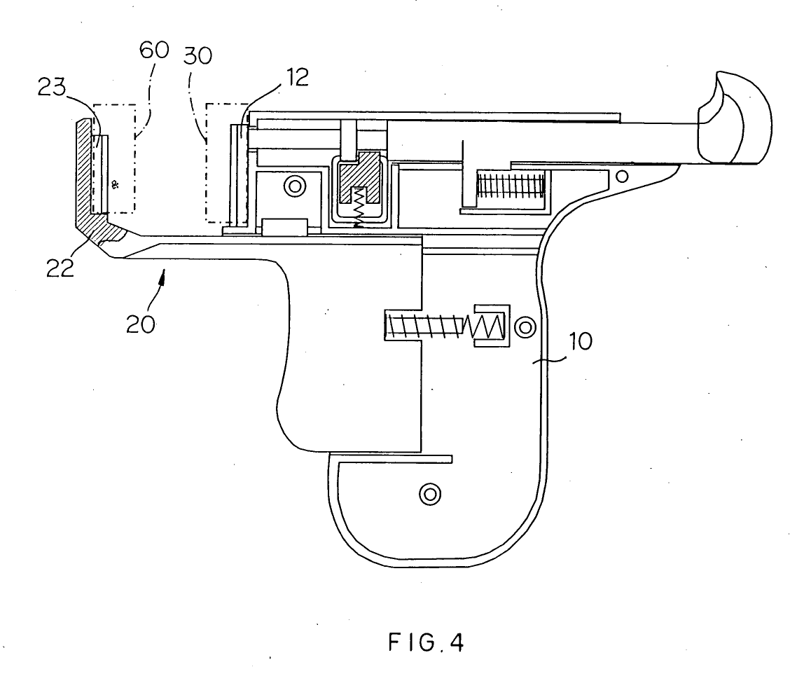

Referring to FIGS. 1 to 4, an ear piercing device in accordance with a preferred embodiment of the invention is shown. The ear piercing device comprises a housing 10 shaped as the butt of a pistol. The housing 10 comprises a vertical first groove 12 on a front end and a horizontal trough 11 below the first groove 12.

The ear piercing device further comprises a movable jaw carrier 20 including a grip member 21 and a first spring 25 having one end anchored in an internal receptacle (not numbered) and the other end put on a rear shaft of the grip member 21 so as to elastically project the grip member 21 out of a front slot (not numbered) of the housing 10. Thus, the movable jaw carrier 20 is a spring biased member and is adapted to elastically slide. The movable jaw carrier 20 further comprises a top plate (not numbered) partially received in the trough 11, a riser 24 projected out of the plate, the riser 24 being limited to slide back and forth in a first compartment (not numbered) of the housing 10 so as to prevent the movable jaw carrier 20 from further moving outwardly to disengage with the housing 10 when the grip member 21 is pressed in an ear piercing operation as detailed later, a leg 22 projected upward from a front end of the plate, and a second groove 23 on a rear surface of the plate shaped leg 22, the second groove 23 facing the first groove 12.

The ear piercing device further comprises a trigger 40 mounted in a second compartment (not numbered) of the housing 10 and having two side tabs (not numbered) each projected out of either side of the housing 10, and a second spring 41 having one end biased against a bottom of the second compartment (not numbered) and the other end anchored in a bottom hole (not numbered) of the trigger 40 so as to elastically push the trigger 40 upward in an inoperative position of the ear piercing device.

The ear piercing device further comprises a slide bar 50 partially received in an upper horizontal channel (not numbered) of the housing 10 and including a projecting rear end enlargement served as striking handle 51, an annular recess 53 lockingly engaged with a top of the trigger 40, and a third spring 52 having one end biased against an internal member of the housing 10 and the other end put on a lower shaft (not numbered) of the trigger 50 so as to elastically push the slide bar 50 forward to dispose the striking handle 51 at a distance away from a rear top corner of the housing 10 in an inoperative position of the ear piercing device.

The ear piercing device further comprises a jaw assembly 30 including a first jaw 31 and a separate second jaw 60. The second jaw 60 is secured to the rear surface of the leg 22 by sliding two sides into two side grooves of the second groove 23. The second jaw 60 comprises two holed shaped members 62 mounted on a rear surface. The first jaw 31 is secured to the first groove 12 by sliding two sides into two side grooves of the first groove 12. The first jaw 31 comprises two hollow protuberances 33 on a front surface, wherein the protuberances 33 are aligned with the holed shaped members 62, two holes (not numbered) each having a rear end open to a rear surface of the first jaw 31 and a front end in communication with the hollow protuberance 33, and two sterile needles 32 wherein only one needle 32 is mounted in a front opening (not numbered) of the housing 10 in the ear piercing operation as detailed later, the opening being aligned with the channel and the hole of the first jaw 31 respectively.

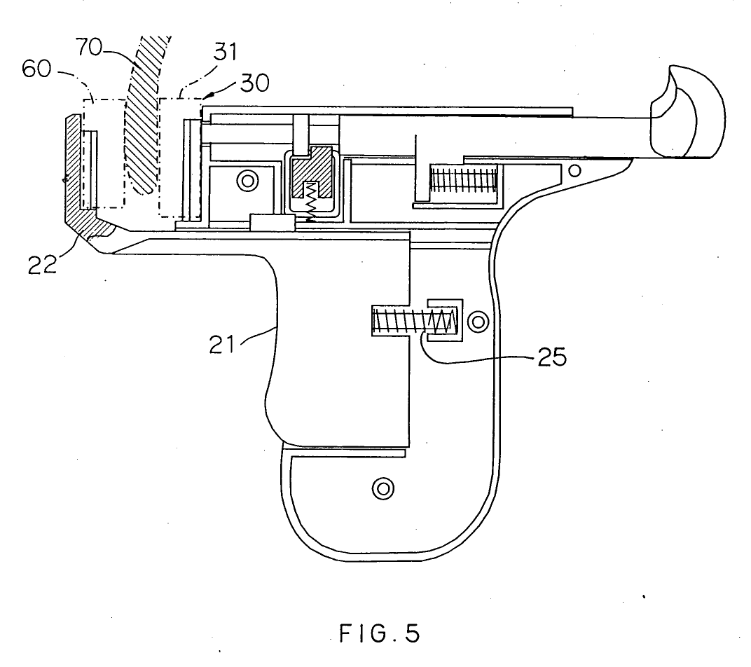

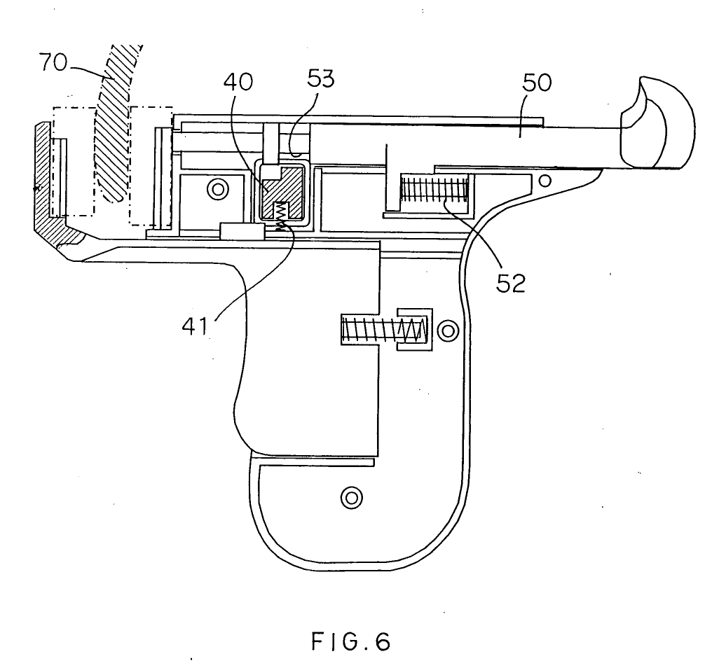

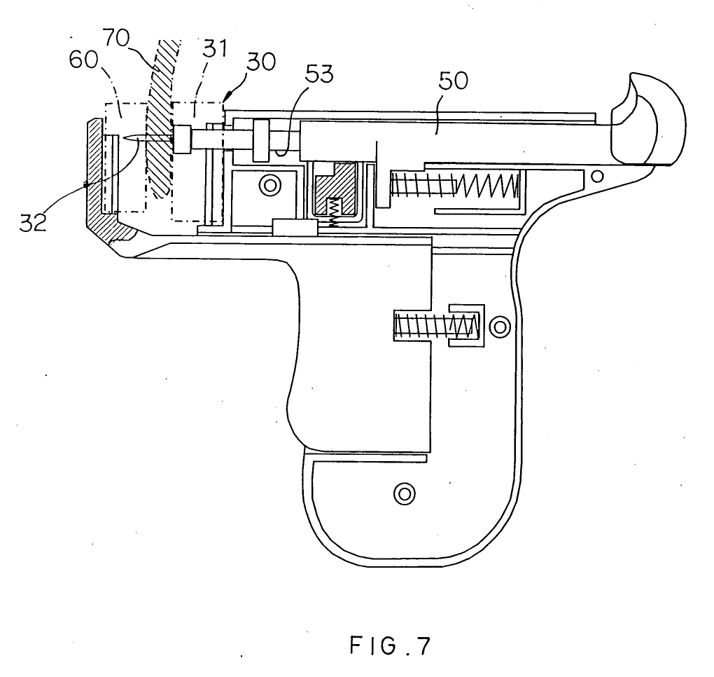

Referring to FIGS. 5, 6, and 7 in conjunction with FIGS. 1 to 4, an ear piercing operation of the invention will be described in detailed below. A person positions the first and second jaws 31 and 61 at both sides of the ear lobe 70 of an ear of a person. Next, press the grip member 21 by one hand to compress the first spring 25 until the ear lobe 70 is clamped by the first and second jaws 31 and 61 with a desired portion of the ear lobe 70 being engaged with one of the protuberances 33. Next, press down the tab(s) of the trigger 40 by the other hand until the top of the trigger 40 clears the recess 53. As such, the slide bar 50 is unlocked. The unlocked slide bar 50 immediately quickly pushes forward to cause its forward end to hammer the head of the needle 32 disposed in the opening as the energized third spring 52 releases its elastic force. The needle 32 thus moves forward along the opening, the hole of the first jaw 31, and the protuberance 33 of the first jaw 31 to insert through the tissue of the ear lobe 70 into the holed shaped member 62. Also, the striking handle 51 is stopped at the rear top corner of the housing 10. As a result, a hole is pierced in the ear lobe 70 by the ear piercing device.

Although the invention has been described for use with a first jaw 31 having two hollow protuberances 33 and a second jaw 61 having two holed shaped members 62 so as to pierce two holes in both ears respectively, it is within the scope of the invention to use a first jaw 31 having a single hollow protuberance 33 and a second jaw 61 having a single holed shaped member 62 so as to pierce a hole in only one ear.

The invention has the following advantages. Both the first and second jaws 31 and 61 are adapted to dispose so as to reduce the risk of spreading diseases as experienced by the well known ear piercing device which has components contacting ear during piercing operation still being used in a next operation. Further, replacement of both the first and second jaws 31 and 61 is quick and simple. Furthermore, the ear piercing device is capable of precisely inserting through the tissue of an ear a sterile needle.

While the invention herein disclosed has been described by means of specific embodiments, numerous modifications and variations could be made thereto by those skilled in the art without departing from the scope and spirit of the invention set forth in the claims.

What is claimed is:

1. An ear piercing device comprising:
a housing including an upper horizontal channel having a rear opening and a front opening, a plurality of compartments, a handle grip, two vertical first grooves on both sides of a front end, and a horizontal trough below the first grooves;
a movable jaw carrier including a spring biased grip member projecting forwardly from the handle grip, a top plate partially received in the trough, a riser projecting from the plate into a first one of the compartments to be limited to slide back and forth therein, a leg projecting upward from a front end of the plate, and two second grooves on both sides of a rear surface of the leg and facing the first grooves;
a spring biased trigger mounted in a second one of the compartments and including two side tabs projecting from the housing, and a top projection;
a spring biased slide bar mounted in the channel and including an enlarged striking handle at a rear end, the striking handle being disposed at a distance away from the rear opening, an intermediate recess lockingly engaged with the projection, and a hammering head at a front end;
a first jaw adapted to releasably secure to the housing by sliding two sides into the first grooves, the first jaw including two hollow protuberances projecting from a front surface, and two holes each having a rear end open to a rear surface and a front end in communication with the protuberance;
a second jaw adapted to releasably secure to the leg by sliding two sides into the second grooves, the second jaw including two yoke members and two holed shaped members each releasably mounted in the yoke members; and
a sterile needle mounted in the front opening and aligned with one hole,
whereby positioning the first and second jaws at both sides of the ear, pressing the grip member to cause the first and second jaws to clamp the ear, and pressing down at least one of the tabs to cause the projection to clear the recess will unlock the slide bar to cause the hammering head to slide to strike the head of the needle so that the needle forcefully moves forward along the front opening, one hole, and one protuberance to insert through the ear into one holed shaped member with the striking handle being stopped at the rear opening.

2. An ear piercing device comprising:
a housing including an upper horizontal channel having a rear opening and a front opening, a plurality of compartments, a handle grip, two vertical first grooves on both sides of a front end, and a horizontal trough below the first grooves;
a movable jaw carrier including a spring biased grip member projecting forwardly from the handle grip, a top plate partially received in the trough, a riser projecting from the plate into a first one of the compartments to be limited to slide back and forth therein, a leg projecting upward from a front end of the plate, and two second grooves on both sides of a rear surface of the leg and facing the first grooves;
a spring biased trigger mounted in a second one of the compartments and including two side tabs projecting from the housing, and a top projection;
a spring biased slide bar mounted in the channel and including an enlarged striking handle at a rear end, the striking handle being disposed at a distance away from the rear opening, an intermediate recess lockingly engaged with the projection, and a hammering head at a front end;
a first jaw adapted to releasably secure to the housing by sliding two sides into the first grooves, the first jaw including a hollow protuberance projecting from a front surface, and a hole having a rear end open to a rear surface and a front end in communication with the protuberance;
a second jaw adapted to releasably secure to the leg by sliding two sides into the second grooves, the second jaw including a yoke member and a holed shaped member releasably mounted in the yoke members; and
a sterile needle mounted in the front opening and aligned with the hole,
whereby positioning the first and second jaws at both sides of the ear, pressing the grip member to cause the first and second jaws to clamp the ear, and pressing down at least one of the tabs to cause the projection to clear the recess will unlock the slide bar to cause the hammering head to slide to strike the head of the needle so that the needle forcefully moves forward along the front opening, the hole, and the protuberance to insert through the ear into the holed shaped member with the striking handle being stopped at the rear opening.
